(19) United States
(12) Patent Application Publication
(10) Pub. No.: US 2014/0128754 A1
(43) Pub. Date: May 8, 2014

(54) MULTIMODAL PHYSIOLOGICAL SENSING FOR WEARABLE DEVICES OR MOBILE DEVICES

(71) Applicants: Michael Edward Smith Luna, San Jose, CA (US); Scott Fullam, Palo Alto, CA (US)

(72) Inventors: Michael Edward Smith Luna, San Jose, CA (US); Scott Fullam, Palo Alto, CA (US)

(73) Assignee: AliphCom, San Francisco, CA (US)

(21) Appl. No.: 13/672,671

(22) Filed: Nov. 8, 2012

(51) Int. Cl. A61B 5/024; A61B 5/00

(52) U.S. Cl. CPC A61B 5/02444; A61B 5/7282; A61B 5/7278
USPC 600/500; 600/300; 600/528

(57) ABSTRACT

Embodiments relate generally to electrical and electronic hardware, computer software, wired and wireless network communications, and wearable computing devices for sensing health and wellness-related physiological characteristics. More specifically, disclosed is a physiological sensor using, for example, acoustic signal energy to determine physiological characteristics in one mode, such as a heart rate, the physiological sensor being disposed in a wearable device (or carried device), and generating data communication signals using acoustic signal energy in another mode. The physiological sensor also can be configured to receive data communication signals. In at least one embodiment, an apparatus includes one or more multimodal physiological sensors configured to receive physiological signals in a first mode and at least generate data communication signals in a second mode. A wearable housing includes the multimodal physiological sensors, and a multimodal physiological sensing device is configured to receive a sensor signal and generate data representing a physiological characteristic.

### FIELD

Embodiments relate generally to electrical and electronic hardware, computer software, wired and wireless network communications, and wearable computing devices for sensing health and wellness-related physiological characteristics. More specifically, disclosed is a physiological sensor using, for example, acoustic signal energy to determine physiological characteristics in one mode, such as a heart rate, the physiological sensor being disposed in a wearable device (or carried device), and generating data communication signals using acoustic signal energy in another mode. The physiological sensor can also be configured to receive data communication signals using acoustic signal energy.

### BACKGROUND

Devices and techniques to gather physiological information, such as a heart rate of a person, while often readily available, are not well-suited to capture such information other than by using conventional data capture devices. Conventional devices typically lack capabilities to capture, analyze, communicate, or use physiological-related data in a contextually-meaningful, comprehensive, and efficient manner, such as during the day-to-day activities of a user, including high impact and strenuous exercising or participation in sports. Further, traditional devices and solutions to obtaining physiological information, such as heart rate, generally require that the sensors remain firmly affixed to the person to employ, for example, low-level electrical signals (i.e., Electrocardiogram (“ECG”) signals). In some conventional approaches, a few sensors are placed directly on the skin of a person while the sensors and the person are to remain relatively stationary during the measurement process. While functional, the traditional devices and solutions to collecting physiological information are not well-suited for use during the course of one's various life activities, nor are traditional devices and solutions well-suited for active participants in sports or over the course of one or more days. Moreover, traditional sensors are delegated to the function of sensing specific characteristics. While functional in the role of sensing, conventional sensors have yet to operate to their capacities.

Thus, what is needed is a solution for data capture devices, such as for wearable devices, without the limitations of conventional techniques.

### BRIEF DESCRIPTION OF THE DRAWINGS

Various embodiments or examples (“examples”) of the invention are disclosed in the following detailed description and the accompanying drawings:

FIG. 1 illustrates an example of a multimodal physiological sensing device disposed in a wearable data-capable band, according to some embodiments;

FIG. 2A is a diagram depicting examples of positions at which a piezoelectric transducer can be disposed, according to some examples;

FIG. 2B is a diagram depicting examples of devices in which a heart rate signal generator and a piezoelectric transducer, and their components, can be disposed or distributed among, according to some examples;

FIGS. 3A to 3C depict a wearable device including a piezoelectric transducer in various configurations, according to some embodiments;

FIGS. 4A and 4B depict a wearable device including an example of an array of piezoelectric transducers, according to some embodiments;

FIGS. 5A and 5B depict control of an array of an array of piezoelectric transducers in a wearable device, according to some embodiments;

FIG. 6 depicts an example of a multimodal piezoelectric signal generator, according to some embodiments;

FIG. 7 is an example flow diagram for multimodal operation of a multimodal physiological sensing device or components thereof, according to some embodiments;

FIG. 8 depicts an example of a multimodal heart rate signal generator, according to some embodiments;

FIG. 9 depicts an example of filtering anomalous heartbeat signals, according to some embodiments; and

FIG. 10 illustrates an exemplary computing platform disposed in or used in association with a wearable device in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

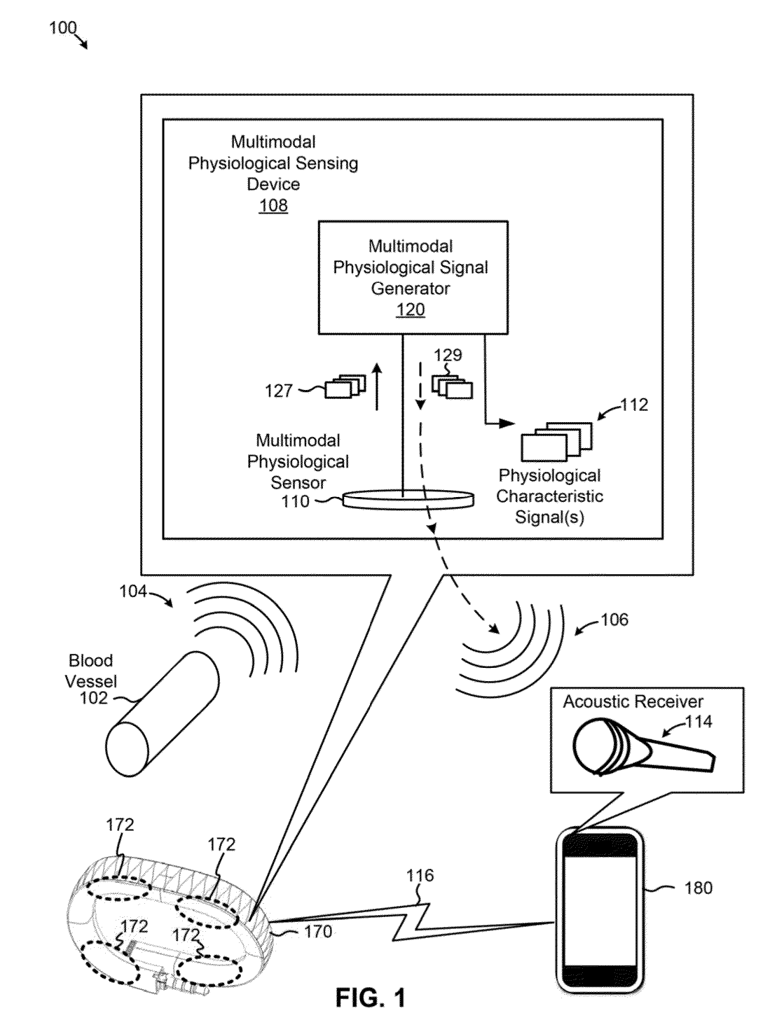

FIG. 1 illustrates an example of a multimodal physiological sensing device disposed in a wearable data-capable band, according to some embodiments. Diagram 100 depicts multimodal physiological sensing device 108 configured to generate one or more physiological characteristic signals in a sensing mode, and to generate and/or receive a data communication signal in a communications mode. For example, multimodal physiological sensing device 108 can sense a heartbeat to generate a physiological characteristic signal, such as a heart rate, in one mode, whereas multimodal physiological sensing device 108 can generate, for example, acoustic data signals with which to transmit data in different mode. As shown, multimodal physiological sensing device 108 includes a multimodal physiological sensor 110 and a multimodal physiological signal generator 120. Multimodal physiological sensor 110 is configured to sense signals, such as physiological signals, associated with a physiological characteristic during one mode. Thus, multimodal physiological sensor 110 can be disposed adjacent to a source of physiological signals 104, such as adjacent to a blood vessel 102, to determining physiological characteristics. Examples of physiological signals 104 include signals representing or including physiological characteristics, such as heart rate, respiration, and other detectable physiological characteristics. Moreover, multimodal physiological sensor 110 is configured to generate data communication signals to transmit data from a wearable device in which multimodal physiological sensor 110 is disposed. Examples of data communication signals include acoustic signals 106 (e.g., with data encoded therein), as well as radio signal, optical signals, electrical signals, etc. In various embodiments, multimodal physiological sensing device 108 can include a single sensor 110 or can include any number multimodal physiological sensors 110 (e.g., an array of such sensors). As used herein, the term “multimodal sensor” can refer, at least in some embodiments, to any device, mechanism, and/or function that is configure to perform a sensing function and at least one other function, such as a communication function.

According to some embodiments, multimodal physiological sensor 110 is a piezoelectric sensor (e.g., a piezoelectric transducer) configured to receive, for example, acoustic energy in a sensing mode, and further configured to generate piezoelectric signals (e.g., electrical signals) in a communication mode. In the example shown, piezoelectric sensor 110 is configured to receive acoustic signal 104 that includes heart-related information. Acoustic signal 104 can propagate through at least human tissue as, for example, one or more sound energy waveforms. Such sound energy signals can originate from either a beating heart (e.g., via a blood vessel 102) or blood pulsing through blood vessel 102, or both. In a sensing mode, piezoelectric sensor 110 converts the acoustic energy of acoustic signal 104 into piezoelectric signals 127 including data representing physiological characteristics, which, in turn, are transmitted to multimodal physiological signal generator 120. Multimodal physiological signal generator 120 converts piezoelectric signals 127 into one or more physiological characteristic signals 112. In a communication mode, piezoelectric transducer 110 (or piezoelectric transducer) is configured to convert piezoelectric signals 129 from multimodal physiological signal generator 120 into one or more data communication signals 106, which can be based on acoustic energy.

In some embodiments, piezoelectric transducer 110 can operate as either a skin surface microphone (“SSM”), or a portion thereof, in a sensing mode. An SSM is configured to receive acoustic energy originating from human tissue rather than airborne acoustic sources that otherwise produce acoustic energy waveforms to propagate through the medium of air. A portion of the SSM is configured to contact (directly or indirectly) human tissue to receive acoustic signals via a contacting portion of the SSM.

In a sensing mode of operation, multimodal physiological signal generator 120 uses sensor 110 to detect and identify, for example, heartbeats, and is further configured to generate physiological characteristic signals 112 representing, for example, a heart rate signal or any other signal including data describing one or more physiological characteristics associated with a user that is wearing or carrying multimodal physiological sensing device 108. In some examples, a heart rate signal or other physiological signals, can be determined (i.e., recovered) from sensed acoustic signals 104 by, for example, comparing the measured acoustic signal against data associated with one or more waveforms of candidate heartbeats. For example, multimodal physiological signal generator 120 can compare, for example, the magnitude of acoustic signal 104 over time against profiles defining characteristics of candidate heartbeats used to identify a heartbeat. A profile can be a data file that defines, describes or otherwise includes characteristics of heartbeats (e.g., in terms of magnitude, timing, pattern reoccurrence, etc.) against which measured data can be compared to determine whether a captured signal portion relates to a heartbeat, according to some embodiments.

In a communication mode of operation, multimodal physiological signal generator 120 uses sensor 110 to generate acoustic signals to communicate data. In a first subset of implementations, a piezoelectric sensor 110 (as sensor 110) can generate audible acoustic signals to serve as alerts or notifications. As used herein, the term “audible” includes frequencies generally between 20 Hz and 16 kHz. In some instances, audible acoustic signals include frequencies up to 20 kHz. In a second subset of implementations, piezoelectric sensor 110 can generate ultrasonic acoustic signals to serve as data communication signals. As used herein, the term “ultrasonic” includes frequencies generally above 20 kHz. Therefore, multimodal physiological signal generator 120 can establish an acoustic data link to form one- or two-way communications. For example, acoustic data communication signal 106 can transmit modified audio waveforms for propagating to, for example, an acoustic receiver 114 (e.g., a microphone) for receiving the data communication signal 106 into a mobile computing device or phone 180. Further, multimodal physiological signal generator 120 can be configured to generate acoustic data communication signal(s) 106 based on a variety of data-encoding techniques. For example, data can be modulated onto acoustic carrier waves based on amplitude and/or frequency modulation.

Note that wearable device 170 can be removed from a wrist, thereby removing a portion of a user's body from blocking or attenuating acoustic data communication signal transmission, such as acoustic data communication signal 116. In some embodiments, piezoelectric sensor 110 (or piezoelectric transducer) operates as both a transmitter and a receiver of acoustic data communication signals 116. For example, in one stage of communication, piezoelectric transducer 310 operates as a transmitter, and in another stage piezoelectric transducer 310 operates as a receiver. Therefore, multimodal physiological signal generator 120 can exchange data (e.g., ultrasonically) with device 180. In some examples, the receiving state of communications and the transmission stage of communications can be associated with different modes.

In some embodiments, multimodal physiological sensing device 108 can be disposed in a wearable device 170. Further, piezoelectric sensor 110, as a multimodal physiological sensor, can be disposed at approximate portions 172 of wearable device 170. In some cases, piezoelectric sensor 110 is disposed in approximate portions 172, which are more likely to be adjacent a radial or ulnar artery than other portions. In some instances, approximate portions 172 provide relatively shorter distances through which acoustic signals propagate from a source to piezoelectric sensor 110. Further, the housing of wearable device 170 can encapsulate, or substantially encapsulate, piezoelectric sensor 110. Thus, piezoelectric sensor 110 can have a portion that is disposed external to the housing of wearable device 170 to contact a skin of a wearer. Or, piezoelectric sensor 110 can be disposed in wearable device 170, which can be formed, at least partially, using an encapsulant that has an acoustic impedance that is equivalent to or is substantially similar to that of human tissue. While wearable device 170 is shown to have an elliptical-like shape, it is not limited to such a shape and can have any shape. Note that multimodal physiological sensing device 108 is not limited to being disposed adjacent blood vessel 102 in an arm, but can be disposed on any portion of a user's person (e.g., on an ankle, ear lobe, behind an ear (i.e., at or near a temporal artery), around a finger or on a fingertip, etc.).

In view of the foregoing, the functions and/or structures of piezoelectric transducer 110 and physiological information generator 120, as well as their components, can facilitate the sensing of physiological characteristics, including heart rate, in situ or during which a user is engaged in physical activity. With the use of piezoelectric sensors/transducers as described herein, electrical signals need not be sensed in human tissue as can be the case in ECG monitoring and bioimpedance sensing. Thus, sensing bio-electric signals need not be at issue when considering proximity to the source of physiological characteristic. Piezoelectric sensor 110 can be used to sense via acoustic signal 104 as a heart-related signal. At least in some instances, the acoustic energy of heart-related signals can propagate through human tissue and/or a vascular system for relatively lengthy distances (e.g., through a limb or the body generally). Further, a piezoelectric sensor can provide a sensing function and a communication function, according to some embodiments. As such, dedicated devices for each of the sensing and communication functions need not be required, thereby conserving space and resources.

In some embodiments, physiological sensor 110 can any suitable structure and sensor for picking up and transferring signals, regardless of whether the signals are electrical, magnetic, optical, pressure-based, physical, acoustic, etc., according to various embodiments. For example, sensor 110 can be configured to operate as a pressure-sensitive sensor to detect displacements, for example, in human tissues (e.g., pulse waves originating in a body) or other pressures applied to wearable device 170. According to some embodiments, physiological sensor 110 can be configured to couple acoustically to a target location, or by other means (e.g., electrically, optically, mechanically, etc.) associated with the type of sensor used.

Piezoelectric sensor 110 can form a skin surface microphone (“SSM”), or a portion thereof, according to some embodiments. An SSM can be an acoustic microphone configured to enable it to respond to acoustic energy originating from human tissue rather than airborne acoustic sources. As such, an SSM facilitates relatively accurate detection of physiological signals through a medium for which the SSM can be adapted (e.g., relative to the acoustic impedance of human tissue). Examples of SSM structures in which piezoelectric sensors can be implemented (e.g., rather than a diaphragm) are described in U.S. patent application Ser. No. 11/199,856, filed on Aug. 8, 2005. As used herein, the term human tissue can refer to, at least in some examples, as skin, muscle, blood, or other tissue. In some embodiments, a piezoelectric sensor can constitute an SSM.

In some embodiments, wearable device 170 can be in communication (e.g., wired or wirelessly) via a communication link 116 with a mobile device 180, such as a mobile phone or computing device. According to some embodiments, communication link 116 can be established using acoustic signals 106. Mobile device 180, or any networked computing device (not shown) in communication with wearable device 170 or mobile device 180, can provide at least some of the structures and/or functions of any of the features described herein. As depicted in FIG. 1 and subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1 (or any subsequent figure) can represent one or more algorithms. Or, at least one of the elements can represent a portion of logic including a portion of hardware configured to provide constituent structures and/or functionalities.

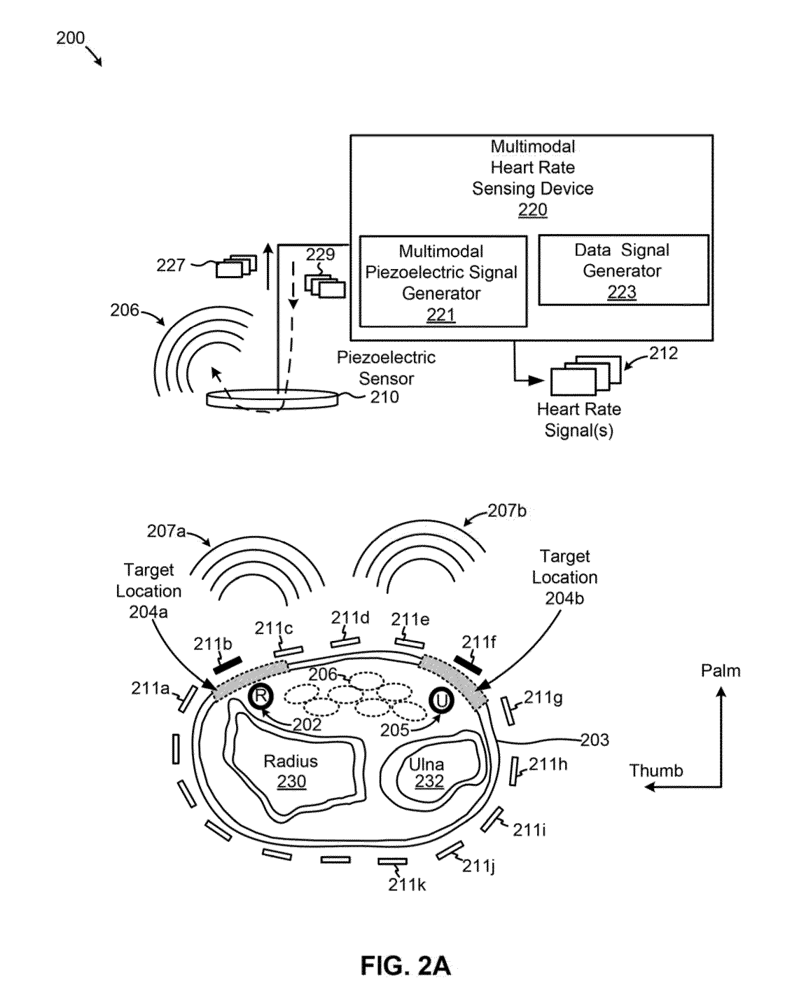

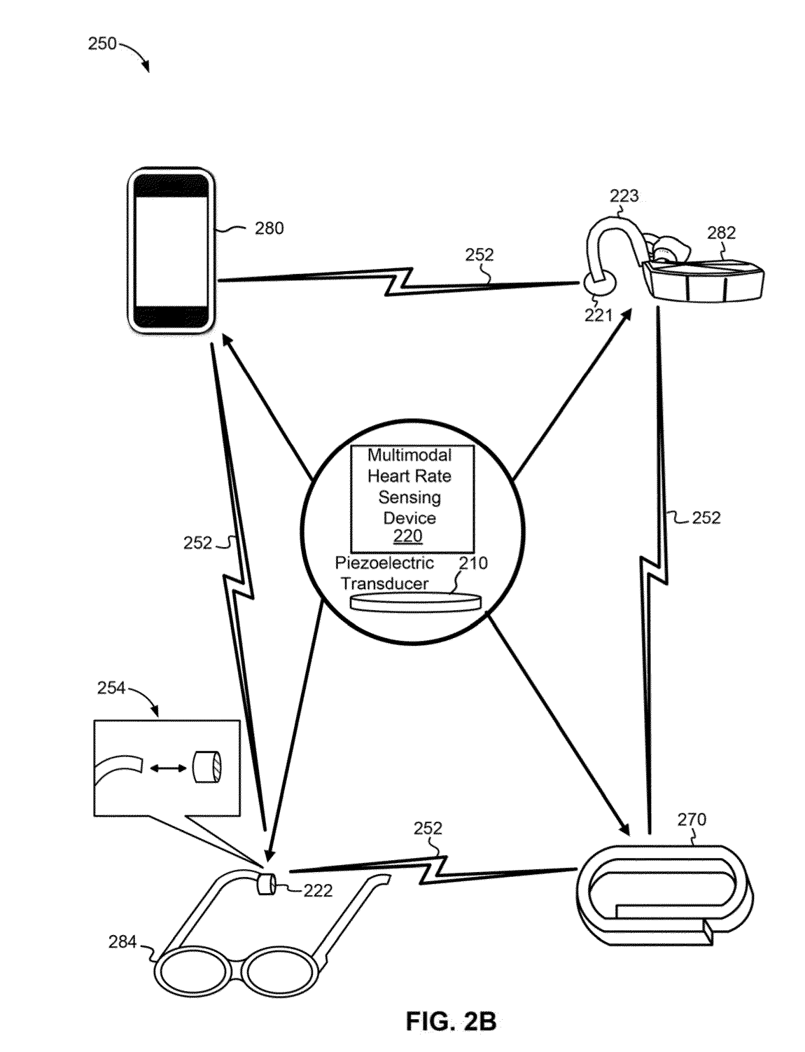

FIG. 2A is a diagram depicting examples of positions at which a piezoelectric transducer can be disposed, according to some examples. Diagram 200 depicts a multimodal heart rate sensing device 220 configured to sense physiological signals, such as acoustic heart-related signals 207a and 207b, and further configured to generate physiological characteristics signals, such as heart rate signals 212, as well as acoustic data communication signals 206. As shown, multimodal heart rate sensing device 220 includes a multimodal piezoelectric signal generator 221 and data signal generator 223. Multimodal piezoelectric signal generator 221 is configured to generate heart rate signals 212 specifying a heart rate for a user based on piezoelectric signals 227 received from piezoelectric transducer 210. Further, data signal generator 223 can cause acoustic data communication signals 206 to be generated, for example, by piezoelectric transducer 210, based on piezoelectric signals 229 transmitted from multimodal heart rate sensing device 220.

Diagram 200 further depicts positions at which piezoelectric transducer 210 may be placed. In particular, positions 211a to 211k represent positions at which piezoelectric transducer 210 can be disposed in a wearable device that is worn on or about a wrist 203 of a user. Note that the terms sensor and transducer can be used equivalently, according to some specific embodiments. In the cross-sectional view shown in FIG. 2A, positions 211a, 211b, 211c, 211d, 211e, 211f, 211g, 211h, 211l, 211j, and 211k, among others, describe positions at which piezoelectric transducer 210 can be disposed about wrist 203 (or the forearm). The cross-sectional view of wrist 203 also depicts a radius bone 230, an ulna bone 232, flexor muscles/ligaments 206, a radial artery (“R”) 202, and an ulna artery (“U”) 205. Radial artery 202 is at a distance 201 (regardless of whether linear or angular) from ulna artery 205. Distance 201 may be different, on average, for different genders, based on male and female anatomical structures. In some cases, piezoelectric transducer 210 (and/or the ability of acoustic signals to propagate through human tissue) can obviate a requirement for a specific placement of piezoelectric transducer 210 due to different anatomical structures based on gender, preference of the wearer, or any other issue that affects placement of piezoelectric transducer 210 that otherwise may not be optimal.

A target region can be adjacent to a source of a physiological characteristic, such as a blood vessel, with which an acoustic signal can be captured and analyzed to identify one or more physiological characteristics. The target region can reside in two-dimensional space, such as an area on the skin of a user adjacent to the source of the physiological characteristic, or in three-dimensional space, such as a volume that includes the source of the physiological characteristic. According to some embodiments, target locations 204a and 204b represent optimal areas (or volumes) at which to measure, monitor and capture data related to acoustic physiological signals, such as acoustic heart-related signals 207a and 207b propagating from radial artery 202 and ulna artery 205, respectively. In particular, target location 204a represents an optimal area adjacent radial artery 202 to pick up acoustic signals 207a originating from artery 202, whereas target location 204b represents another optimal area adjacent ulna artery 205 to pick up other acoustic signals 207b originating from artery 205. For example, positions 211b and 211f can receive acoustic signals 207a and 207b associated with radial artery 202 and ulna artery 205, respectively without intervening tissues masses, such as flexor muscles/ligaments 206 or bones 230 and 232. As used herein, the term “target location” can, for example, refer to a region in space from which a physiological characteristic can be determined. More or fewer piezoelectric transducers 210 can be used.

In some embodiments, multiple piezoelectric transducers 210 can be arranged in an array and disposed in any of the positions 211a, 211b, 211c, 211d, 211e, 211f, 211g, 211h, 211l, 211j, and 211k. For example, a first piezoelectric transducer can be disposed at position 211b and a second piezoelectric transducer can be disposed at position 211f to sense acoustic signals from radial artery 202 and ulna artery 205, respectively.

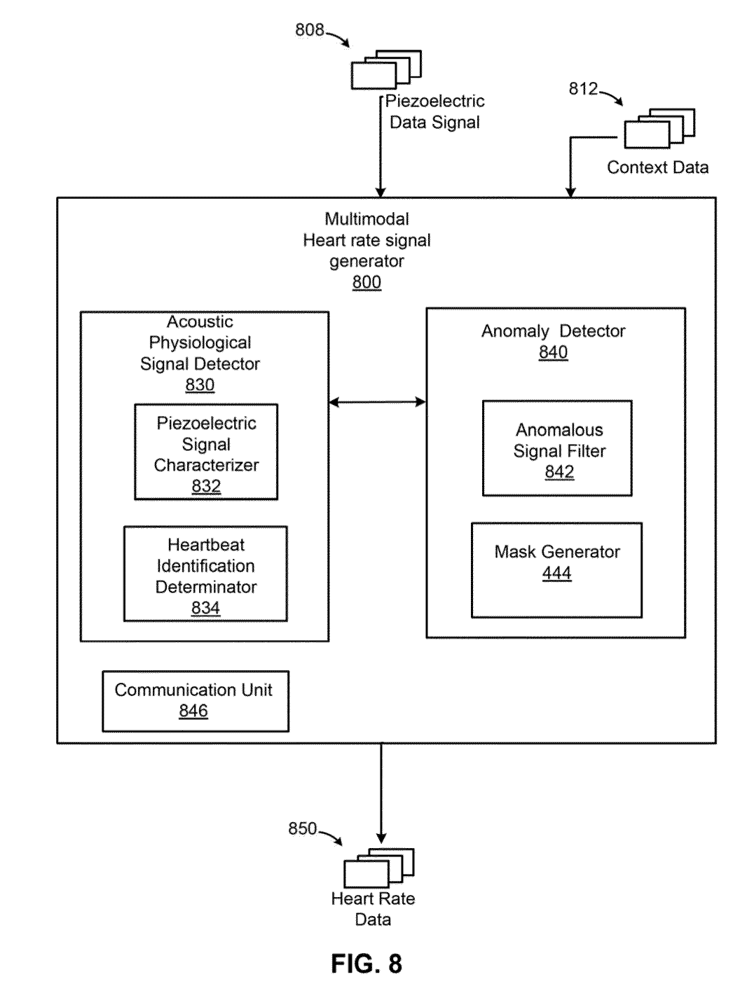

FIG. 2B is a diagram depicting examples of devices in which a multimodal heart rate sensing device and a piezoelectric transducer, and their components, can be disposed or distributed among, according to some examples. Diagram 250 depicts examples of devices (e.g., wearable or carried) in which multimodal heart rate sensing device 220 and piezoelectric transducer 210 can be disposed include, but are not limited to, a mobile phone 280, a headset 282, eyewear 284, and a wrist-based wearable device 270. In some instances, multimodal heart rate sensing device 220 and/or piezoelectric transducer 210 can be implemented as an acoustic heart rate sensor 221 or 222. Acoustic heart rate sensor 221 is disposed on or at an earloop 223 of headset 282 (e.g., a Wi-Fi headset, a Bluetooth® communications headset, or other types of communications) to position piezoelectric transducer 210 adjacent to human tissue (e.g., behind an ear). Acoustic heart rate sensor 222 can be disposed on or at the ends of eyewear 284 (e.g., at temple tips that extend over an ear) to position piezoelectric transducer 210 adjacent to human tissue (e.g., behind an ear). Acoustic heart rate sensors, such as sensor 222, can be configured to detach and attach, as shown in view 254, to any of the devices described. Further, acoustic heart rate sensors described in FIG. 2B can include a communications unit, such as described in FIG. 8, to establish communications links 252 (e.g., wireless or acoustic data links) to communicate heart-related data signals among the devices. While piezoelectric transducer 210 is described as being disposed in association with devices 280, 282, 270, and 284, FIG. 2B is not intended to be limiting. For example, piezoelectric transducer 210 and/or multimodal heart rate sensing device 220 can be implemented internally to a user's body.

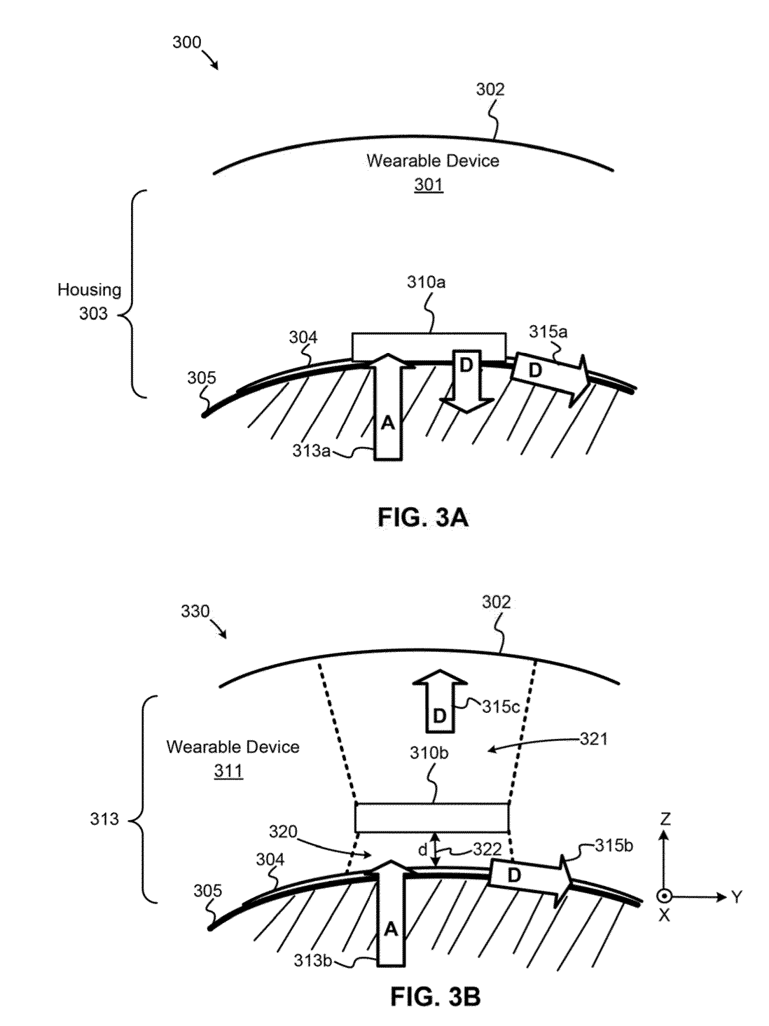

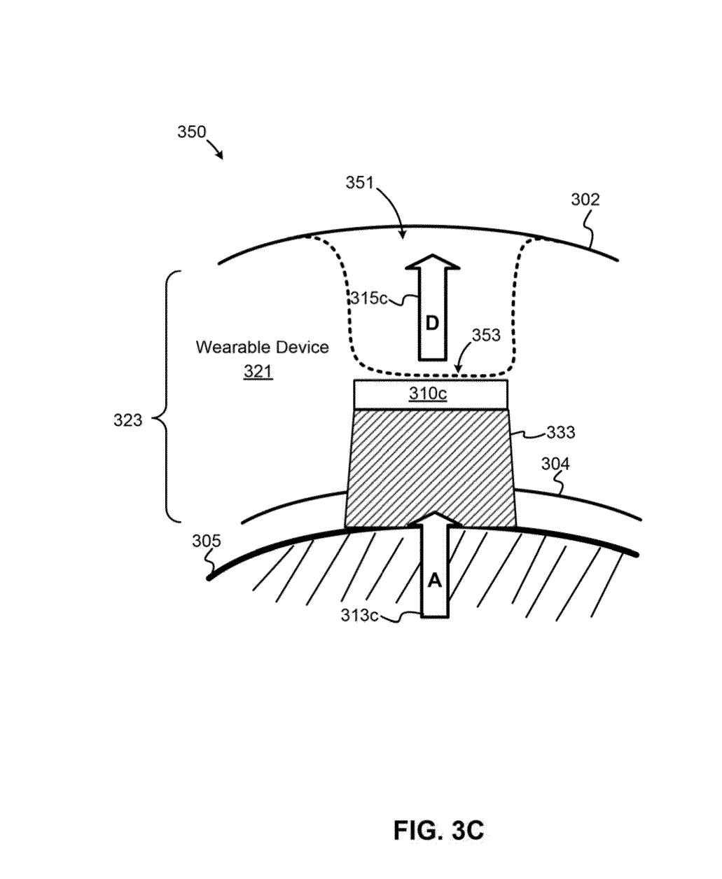

FIGS. 3A to 3C depict a wearable device including a piezoelectric transducer in various configurations, according to some embodiments. Diagram 300 of FIG. 3A depicts a wearable device 301, which has an outer surface 302 and an inner surface 304. In some embodiments, wearable device 301 includes a housing 303 configured to position a piezoelectric transducer 310a (or an SSM including a piezoelectric transducer) to receive an acoustic signal (“A”) 313a originating from human tissue, such as skin surface 305, in a first mode. As shown, at least a portion of piezoelectric transducer 310a is formed external to surface 304 of wearable housing 303. The exposed portion of the piezoelectric transducer is configured to contact skin 305 (directly or indirectly). Further, piezoelectric transducer 310a can be configured to generate acoustic data communication signals (“D”) 315a in a second mode. Acoustic data communication signals (“D”) 315a are depicted as being transmitted to an external environment out from between surface 304 and skin 305. In some instances, an acoustic data communication signal 315a can be transmitted into skin 305 (e.g., to be picked up by another sensor, such as an SSM, adjacent to piezoelectric transducer 310a or at any position on the user's body). Note that wearable device 301 can be removed from a wrist, thereby removing skin 305 from blocking or attenuating acoustic data communication signals 315a. In some embodiments, piezoelectric transducer 310a operates as both a transmitter and a receiver of acoustic data communication signals 315a. For example, in one stage of communication, piezoelectric transducer 310a operates as a transmitter, and in another stage piezoelectric transducer 3100a operates as a receiver.

Diagram 330 of FIG. 3B depicts a wearable device 311, which has an outer surface 302 and an inner surface 304. In some embodiments, wearable device 311 includes a housing 313 configured to position a piezoelectric transducer 310b (or an SSM including a piezoelectric transducer) to receive an acoustic signal (“A”) 313b originating from human tissue, such as skin surface 305, in a sensing mode. As shown, piezoelectric transducer 310b is disposed in wearable housing 313 at a distance (“d”) 322 from inner surface 304. Material, such as an encapsulant, can be used to form wearable housing 313 to reduce or eliminate exposure to elements in the environment external to wearable device 311.

In some embodiments, a portion of an encapsulant or any other material can be disposed or otherwise formed at region 320 to facilitate propagation of an acoustic signal to the piezoelectric transducer. The material and/or encapsulant can have an acoustic impedance value that matches or substantially matches the acoustic impedance of human tissue and/or skin. Values of acoustic impedance of the material and/or encapsulant can be described as being substantially similar to the human tissue and/or skin when the acoustic impedance of the material and/or encapsulant varies no more than 60V % of that of human tissue or skin, according to some embodiments. Examples of materials having acoustic impedances matching or substantially matching the impedance of human tissue can have acoustic impedance values in a range that includes 1.5×10⁶ Pa×s/m (e.g., an approximate acoustic impedance of skin). In some examples, materials having acoustic impedances matching or substantially matching the impedance of human tissue can provide for a range between 1.0×10⁶ Pa×s/m and 1.0×10⁷ Pa×s/m. Note that other values of acoustic impedance can be implemented to form one or portions of housing 313. In some examples, the material and/or encapsulant can be formed to include at least one of silicone gel, dielectric gel, thermoplastic elastomers (TPE), and rubber compounds, but is not so limited. As an example, the housing can be formed using Kraiburg TPE products. As another example, housing can be formed using Sylgard® Silicone products. Other materials can also be used.

Further, piezoelectric transducer 310b can be configured to generate acoustic data communication signals (“D”) 315b in a communications mode. Acoustic data communication signals 315b are depicted as transmitted to an external environment out from between surface 304 and skin 305. In some instances, an acoustic data communication signal 315c can be transmitted through a portion 321 of wearable housing 313. In some embodiments, a portion of an encapsulant or any other material can be disposed or otherwise formed at portion 321 to facilitate propagation of an acoustic signal from piezoelectric transducer 310b to an external environment either in the Z-direction (as shown) or in X-direction (not shown), such as through a side surface of wearable housing 313. The material and/or encapsulant in portion 321 can have an acoustic impedance value that facilities transmission to the external environment.

Diagram 350 of FIG. 3C depicts a wearable device 321, which has an outer surface 302 and an inner surface 304. In some embodiments, wearable device 321 includes a housing 323 configured to position a piezoelectric transducer 310c (or an SSM including a piezoelectric transducer) to receive an acoustic signal (“A”) 313c originating from human tissue, such as skin surface 305, one mode. A portion of piezoelectric transducer 310c is configured to receive acoustic signals 313c via a coupler 333 from skin 305. As shown, piezoelectric transducer 310c is disposed in wearable housing 313 at a distance from inner surface 304. In this example, coupler 333 is disposed between piezoelectric transducer 310c and inner surface 304 and is configured to contact skin 305 at one end and to communicate acoustic signals to piezoelectric transducer 310c at the other end. Coupler 333 can be composed of an equivalent material to that described in FIG. 3B to facilitate propagation of acoustic signal 313c to piezoelectric transducer 310c.

Further, piezoelectric transducer 315c can be configured to generate acoustic data communication signals (“D”) 315c in another mode. Acoustic data communication signals 315c are depicted as transmitted to an external environment out from between surface 302 and a portion of piezoelectric transducer 310c (e.g., through a relatively then portion 353 of wearable housing 323. In some instances, a cavity 351 is formed within wearable device 323. Portion 353 is formed to have a dimension (e.g., thinness) configured to facilitate transmission of acoustic data communication signals 315c to the external environment.

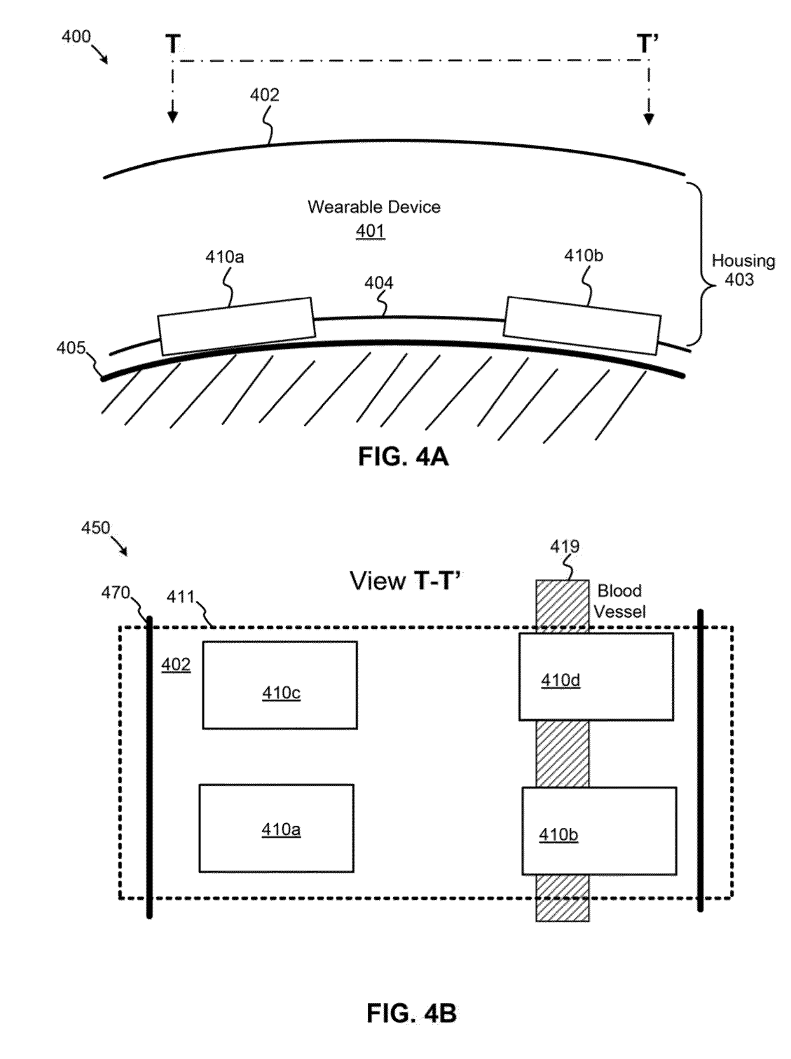

FIGS. 4A and 4B depict a wearable device including an example of an array of piezoelectric transducers, according to some embodiments. Diagram 400 of FIG. 4A depicts a wearable device 401, which has an outer surface 402 and an inner surface 404. In some embodiments, wearable device 401 includes a housing 403 configured to position an array of piezoelectric transducers, including piezoelectric transducers 410a and 410b (or any other like sensor) to receive an acoustic signal originating from human tissue, such as skin surface 405, in a first mode. As shown, at least a portion of piezoelectric transducer 410a is formed external to surface 404 of wearable housing 403. The exposed portion of the piezoelectric transducer can be configured to contact skin 405 (directly or indirectly). To illustrate, consider that including piezoelectric transducers 410a and 410b can be configured to be disposed at or adjacent a radial artery and an ulna artery, respectively. Further, piezoelectric transducers 410a and 410b can be configured to generate acoustic data communication signals in a second mode. For example, one or more acoustic data communication signals can be transmitted to an external environment (e.g., out from between surface 404 and skin 405, or through housing 403).

Diagram 450 of FIG. 4B depicts a top view (T-T′) of an example of an array of piezoelectric transducers depicted in FIG. 4A. As shown, wearable device 411 having an outer surface 402 is disposed about a user wrist 470. An array of piezoelectric transducers is shown to include piezoelectric transducers 410a and 410b of FIG. 4A, as well as piezoelectric transducers 410c and 410d. Subsets of any number or type of piezoelectric transducer can be configured to perform a sensing function and/or a communications function. For example, piezoelectric transducers 410b and 410d can be configured disposed adjacent a blood vessel 419, each of which can perform either a sensing function or a communications function, or both. As another example, piezoelectric transducers 410a and 410c can be configured disposed a distance from blood vessel 419, each of which can perform at least a communications function. Further, piezoelectric transducers 410a and 410c can each be differently configured to generate different acoustic data communication signals (e.g., at different frequencies). In other examples, piezoelectric transducers 410a and 410c can be configured disposed adjacent a blood vessel (not shown), such if wearable device 411 is disposed on the other wrist. In this case, piezoelectric transducers 410a and 410c can perform either a sensing function or a communications function, or both.

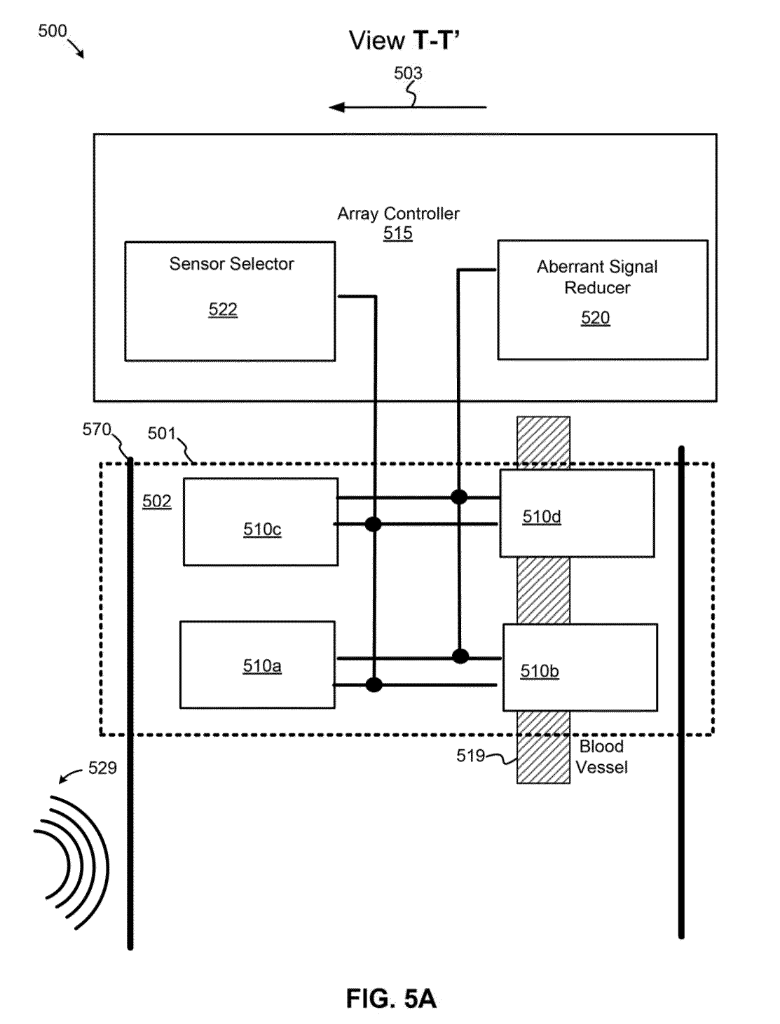

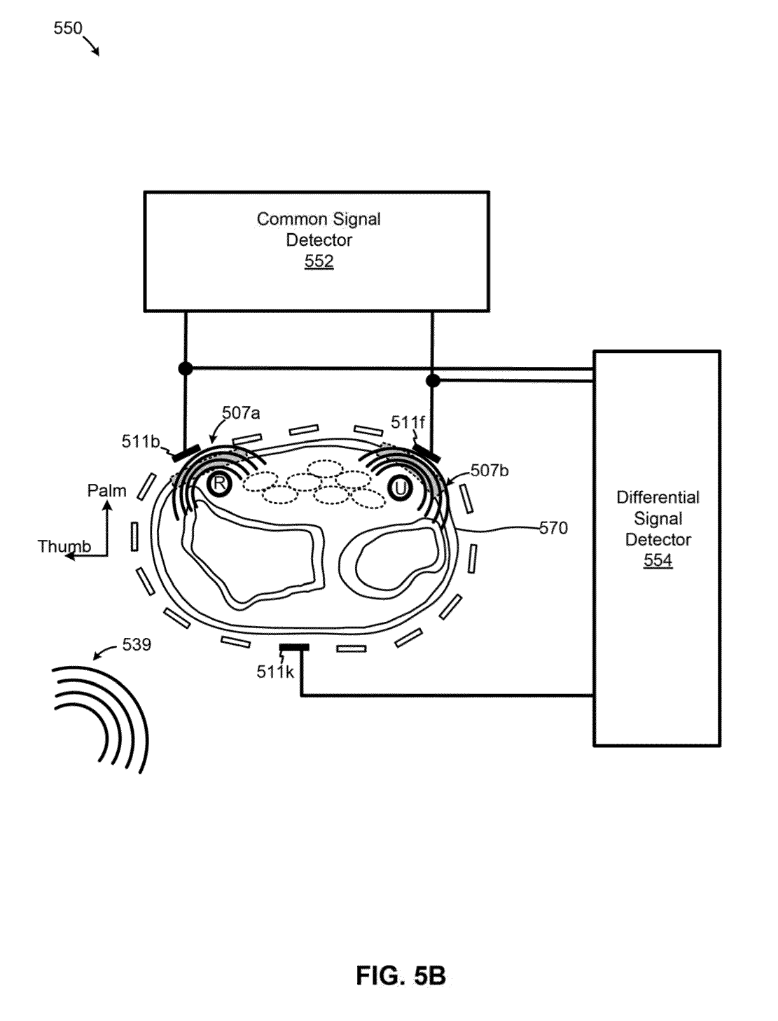

FIGS. 5A and 5B depict control of an array of an array of piezoelectric transducers in a wearable device, according to some embodiments. Diagram 500 of FIG. 5A is a top view depicting a wearable device 501 including an array controller 515 configured to control array of including piezoelectric transducers 510a, 510b, 510c and 510d. Wearable device 501 is shown to have an outer surface 502, and that wearable device 501 is disposed about a wrist 570 (or any other limb or extremity). Array controller 515 includes a sensor selector 522 is configured to select a subset of piezoelectric transducers, and is further configured to use the selected subset of piezoelectric transducers to acquire physiological characteristics in association with a target location, according to some embodiments.

In some embodiments, sensor selector 522 can be configured to determine (periodically or aperiodically) whether a subset of piezoelectric transducers includes optimal piezoelectric transducers for acquiring a sufficient representation of the one or more physiological characteristics from an acoustic signal. To illustrate, consider that piezoelectric transducers 510a and 510c may be displaced from the target location when, for instance, wearable device 501 is subject to a displacement 503 in a plane substantially perpendicular to blood vessel 502 (e.g., the wearable device 501 rotates about wrist 570). Displacement 503 of piezoelectric transducers 510a and 510c may cause a decrease of the strength of an acoustic signal generated by blood vessel 519 as the distance between piezoelectric transducers 510a and 510c and blood vessel 519 increases. Displacement of piezoelectric transducers 510a and 510c from the target location, therefore, may degrade or attenuate the acoustic signals retrieved therefrom. While piezoelectric transducers 510a and 510c may be displaced from the target location, other piezoelectric transducers can be displaced to the position previously occupied by piezoelectric transducers 510a and 510c (i.e., adjacent to the target location adjacent blood vessel 519). For example, piezoelectric transducers 510b and 510d may be displaced to a position adjacent to blood vessel 519. In this case, sensor selector 522 operates to determine an optimal subset of piezoelectric transducers, such as piezoelectric transducers 510b and 510d, to acquire via acoustic signals one or more physiological characteristics (e.g., by selecting subsets of piezoelectric transducers receiving the greatest acoustic magnitudes, or the loudest signals). Therefore, regardless of the displacement of wearable device 501 about blood vessel 519, sensor selector 522 can repeatedly determine an optimal subset of piezoelectric transducers for extracting physiological characteristic information from adjacent a blood vessel. For example, sensor selector 522 can repeatedly test subsets in sequence (or in any other manner) to determine which one is disposed adjacent to a target location.

Aberrant signal reducer 520 is configured to reduce or negate acoustic-related signals (or any other noise-related signal) unrelated to the desired acoustic signals (e.g., pulse waves in blood vessel 519). An aberrant signal can include acoustic energy unrelated to the acoustic energy relating to a physiological characteristic (e.g., a heartbeat), which may or may not form a portion of the acoustic signal received by the array of piezoelectric transducers. For example, aberrant acoustic signal 529 can impinge upon or propagate through wrist 570. Examples aberrant acoustic signal 529 include acoustic signals generated by wearable device 510 rotating or sliding on wrist 570 (e.g., scratch-related noises), by a users' fingers typing on a keyboard, by receiving a common sound produced by tapping on wrist 570, or any other similar sounds. Aberrant signal reducer 520 operates to eliminate the magnitude of an aberrant signal component, or to reduce the magnitude of the aberrant signal component relative to the magnitude of the physiological-related signal component, such as a heartbeat, thereby yielding as an output the physiological-related signal component (or an approximation thereto). Thus, aberrant signal reducer 520 can reduce the magnitude of the aberrant signal component by an amount associated with a piezoelectric transducer that is positioned to receiving principally or predominantly the aberrant signal.

FIG. 5B is a diagram 550 depicting example components of aberrant signal reducer of FIG. 5A, according to some embodiments. As shown, an aberrant signal reducer can include one or both of a common signal detector 552 and a differential signal detector 554 disposed in a wearable device about a wrist 570. Common signal detector 552 is coupled to piezoelectric transducers 511b and 511f, which are configured to receive acoustic signals 507a and 507b, respectively. Common signal detector 552 is configured to detect and amplify at least common portions of acoustic signals 507a and 507b that related to a heart-related signal, and is further configured to determine an acoustic signal representative of a heartbeat (e.g., with portions of an aberrant signal component reduced or filtered out). Differential signal detector 552 is coupled to one or both of piezoelectric transducers 511b and 511f and to piezoelectric transducer 511k, which is configured to receive principally or predominantly aberrant acoustic signal 539. Differential signal detector 552 is configured to detect and identify at least different portions of, for example, acoustic signal 507b with aberrant signal components. The detected aberrant acoustic signal 539 is used to remove the aberrant signal components to obtain acoustic signal 507b.

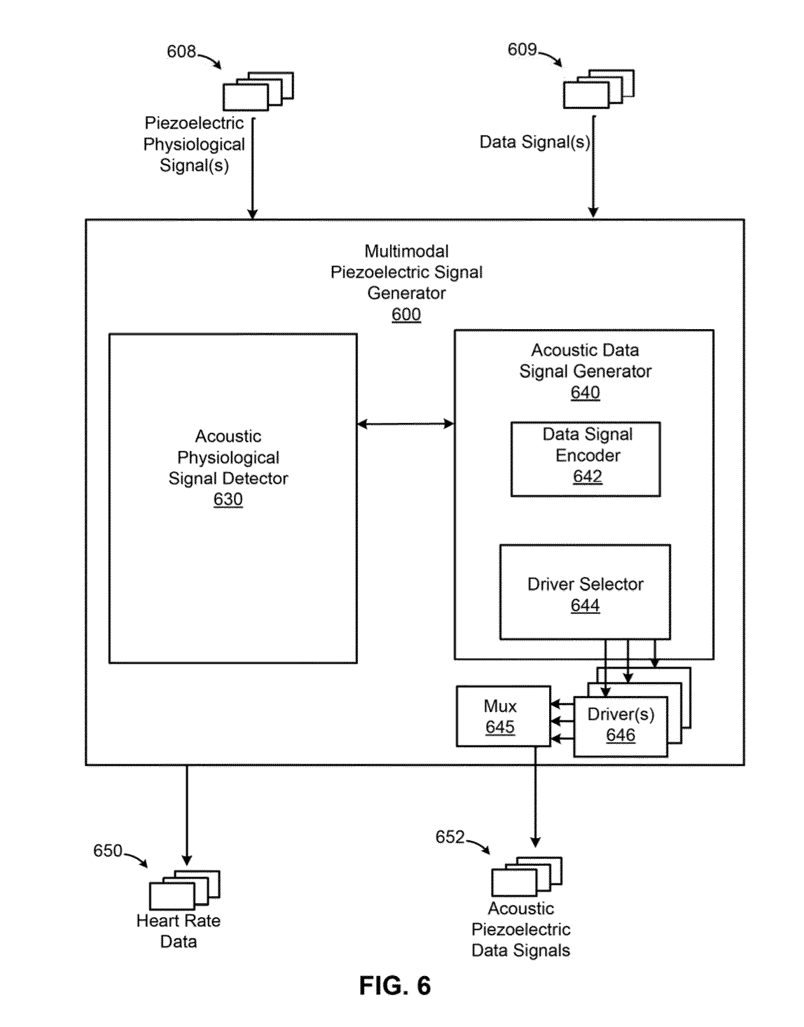

FIG. 6 depicts an example of a multimodal piezoelectric signal generator, according to some embodiments. Multimodal piezoelectric signal generator 600 includes an acoustic physiological signal detector 630 and an acoustic data signal generator 640. Acoustic physiological signal detector 630 is configured to receive at least piezoelectric physiological signals 608 from a piezoelectric transducer and to generate a signal 650 representing a physiological characteristic, such as a heart rate. Examples of acoustic physiological signal detector 630 are discussed in FIG. 8.

Acoustic data signal generator 640 is configured to generate acoustic communication data signals using one or more piezoelectric transducers. Acoustic data signal generator 640 includes a data signal encoder 642, a drive selector 644, one or more drivers 646, and an optional mux 645 to select the drivers 646. Different drivers can driver different piezoelectric transducers that, for example, a different audible frequencies, according to some embodiments. Data signal encoder 642 is configured to receive one or more data signal(s) 609 (e.g., digital signals) and to encode the data in signals 609 to generate encoded data signals, which can be analog forms of the acoustic communications data signals. For example, one or more data signal(s) 609 can include data representing a heart rate of 90 beats per minutes (“bpm”). Drive selector 644 is configured to select one or more drivers 646 to drive one or more piezoelectric transducers in an array of piezoelectric transducers to transmit acoustic data signals 652 to an external environment. In some embodiments, drive selector 644 can select one or more piezoelectric transducers configured to generate audible acoustic signals. Further, drive selector 644 can select one or more piezoelectric transducers configured to generate ultrasonic acoustic signals. In some embodiments, drive selector 644 can select one or more piezoelectric transducers to receive audible or ultrasonic acoustic signals, or the like.

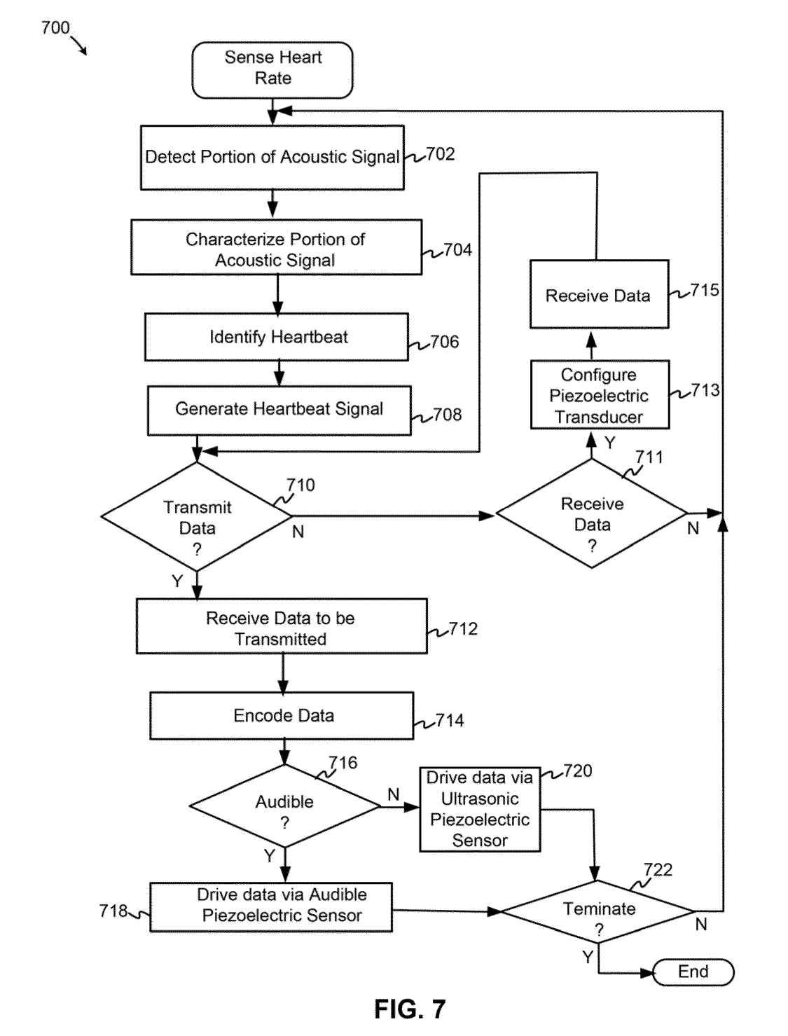

FIG. 7 is an example flow diagram for multimodal operation of a multimodal physiological sensing device or components thereof, according to some embodiments. At 702, flow 700 detects a portion of an acoustic signal (e.g., as a piezoelectric signal portion). At 704, one or more portions of the acoustic signal are characterized to determine whether the portions include heart-related signals. At 706, a heartbeat is identified from the acoustic signal, and a heartbeat signal is generated at 708 to including heartbeat information (e.g., beats per minute, etc.). At 710, a determination is made whether to transmit data. If so, data to be transmitted is received at 712 and encoded at 714. If data is to be transmitted audibly, such a determination is made at 716. If audible, then flow 700 moves to 718 at which audible piezoelectric signals are driven to one or more piezoelectric transducers to generate audible data communication signals. If at 716, an ultrasonic signal is to be generated, then flow 700 moves to 720 at which ultrasonic piezoelectric signals are driven to a piezoelectric transducer to generate ultrasonic data communication signals. Flow 700 moves past 722 if the flow is not to be terminated. Returning back to 710, if a determination is made not to transmit data, flow 700 moves to 711 to determine whether to receive data at 711. If so, then flow 700 moves to 713 at which a piezoelectric transducer is configured to receive data, and data is received at 715. Flow 700 then continues from 710.

FIG. 8 depicts an example of a multimodal heart rate signal generator, according to some embodiments. The diagram of FIG. 8 depicts a multimodal heart rate signal generator 800 that can be disposed in a wearable device or distributed over the wearable device and other devices, such as a mobile computing device or phone. Heart rate signal generator 800 can be configured to receive piezoelectric data signals 808 from a piezoelectric transducer and, optionally, context data 812. Context data 812 includes data describing the context in which a heart rate is being determined. For example, context data 812 includes an age of the user, motion data describing an activity or general level of motion of the user (e.g., whether the user is sleeping, sitting, running a marathon, etc.), a location of the user, and other types of data that can assist determining a heart rate. The age of the user can determine normative or expected heart rates as older users typically have slow heart rates than younger users. This information can assist in excluding anomalous data. Heart rate signal generator 800 also can be configured to generate heart rate data 850 that describes the heart rate of a user.

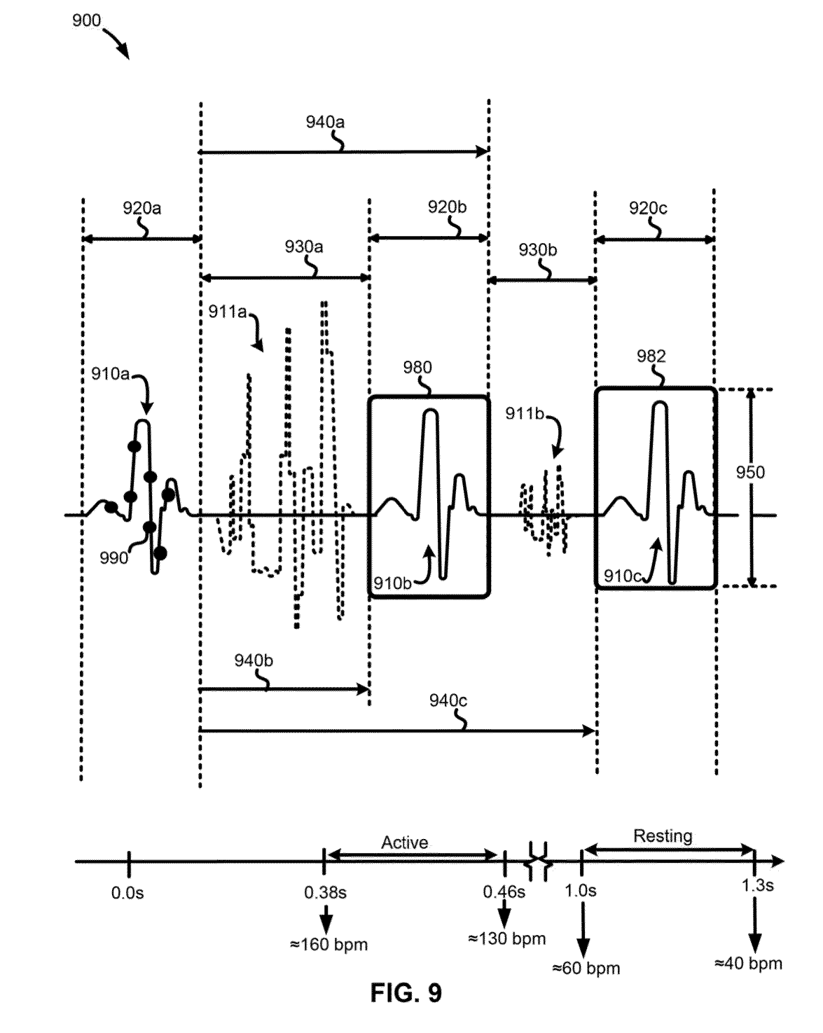

Heart rate signal generator 800 can include one or more of a heart rate processor 830 configured to determine one or more heartbeats constituting a heart rate, and an anomaly detector 840 configured to detect or otherwise exclude data that are unlikely related to a heartbeat. As used herein, the term anomalous data or signals can refer, at least in some examples, to data and/signals that have values that may be inconsistent with expected values describing a range of values associated with candidate heart beats. For example, a candidate heartbeat, such as heart beat 910a of FIG. 9, can be described in terms of one or more data points 990 of FIG. 9 expressing detected signal magnitudes at different times. As a candidate heartbeat, data points 990 (e.g., samples) can represent likely heartbeat characteristics (e.g., magnitudes and timing) that can define expected data points and characteristics of likely heartbeats. These characteristics, when analyzed within certain tolerances, can indicate whether piezoelectric data signals 808 (or portions thereof) indicate a heartbeat, when compared to piezoelectric data signals 808. Referring back to FIG. 8, heart rate processor 830 is configured to compare measured portions of piezoelectric data signal 808 to data files (e.g., profiles) that define characteristics of heartbeats (e.g., in terms of magnitude, timing, pattern reoccurrence, etc.), according to some embodiments.

Heart rate processor 830 can include a piezoelectric signal characterizer 832 and a heartbeat identification determinator 834. Piezoelectric signal characterizer 832 is configured to amplify the piezoelectric data signals and to characterize the values of piezoelectric data signals 808. For example, piezoelectric signal characterizer 832 can determine characteristics of portions of piezoelectric signals to, for example, establish values associated with data points, such as data points 990 of FIG. 9.

Anomaly detector 840 can include an anomalous signal filter 842 and a mask generator 844. Anomalous signal filter 842 is configured to determine which data points 990 (or samples) are considered valid for purposes of determining a heartbeat. For example, data points having magnitudes above an expected magnitude of an acoustic signal generated by a heart-related event likely are not due to pulsing blood (e.g., it is rare that a sudden, instantaneous exertion of the heart occurs). Thus, anomalous signal filter 842 can indicate that data points 990 above a certain magnitude ought not be considered as part of a heartbeat. In some implementations, anomalous signal filter 842 receives the characterized piezoelectric signals from piezoelectric signal characterizer 832.

Mask generator 844 is also configured to mask or otherwise exclude data from heartbeat consideration when determining one or more heartbeats. Mask generator 844 consumes context data 812. For example, older users and younger users are expected to have different heart rates when resting and being active. As such, mask generator 844 excludes from consideration heart rates that occur in other age ranges that need not pertain to the age range in which the user occupies. As another example, mask generator 844 excludes from consideration heart rates that are inconsistent with motion data (e.g., a high heart rate range of 130 to 160 bpm is excluded if motion data suggests that the person is resting or sleeping). Likewise, changes in location due to user-generated to motion (e.g., running) is unlikely to be accompanied by heart rates indicative to sleeping. Therefore, mask generator 844 excludes from consideration heart rates that are below those that define an active person, when, in fact, the user is in motion. Further, mask generator 844 can define windows or intervals within to analyze a next heart beat based on previous samples of heartbeats. As heart rates to do not normally change instantaneously, mask generator 844 can modify the timing when the windows or intervals open to accept data presumed valid and when to exclude other data unlikely to be heart-related. Mask generator 844 is configured to provide heartbeat identification determinator 834 with piezoelectric data samples that have not been masked, whereby heartbeat identification determinator 834 determines a heartbeat and an approximate point in time at which the heart beat occurs. Subsequent heartbeats can be determined relative to the point in time in which an earlier heart beat has been determined. Heartbeat identification determinator 834 can then generate heart rate data 850 that includes a real-time (or near real-time) heart rate. In some embodiments, heart rate signal generator 800 can include a communication unit 846 including hardware, software, or a combination thereof, configured to transmit and receive control and heart-related data to other devices, such as those described in FIG. 2B. Heart rate signal generator 800 and/or anomaly detector 840 can operate individually or cooperatively to determine trend data representing approximate intervals between heartbeats over time. The approximate intervals can change as the user transitions through different levels of activity (e.g., from resting to walking to running).

FIG. 9 depicts an example of filtering anomalous heartbeat signals, according to some embodiments. Diagram 900 of FIG. 9 depicts portions of a piezoelectric signal including portions 910a, 910b, and 910c that include characteristics that predominantly match those of expected heartbeats. In this example, consider that portion 910a is determined to include or represent a valid representation of a heartbeat during interval 920a. In some examples, portion 911a is determined to include amplitudes or magnitudes that exceed an expected magnitude 950. Therefore, anomaly detector 840 can invalidate or mask portion 911a from being considered. Further, portion 911b is determined to include amplitudes or magnitudes that fall below an expected minimum magnitude (not shown), and can be invalidated to remove from consideration. Alternatively, or in addition to the aforementioned, portion 911a can be determine to coincide with interval 930a (e.g., above 160 bpm), and thus can be invalidated (and masked). Portion 911b can occur during intervals 930b, which can be either slower than during active interval 920b or faster than during resting interval 920c. Thus, portion 911b can be invalidated (and masked) if the user's activity does not suggest a heart rate associate with the timing of portion 911b. Mask generator 844 can be further configured to exclude portion 910c when a trend of heartbeat data suggest that the sampling window 980 in which to accept data is from time 940b to time 940a after a heartbeat is detected at 920a (i.e., the user is active). Or, mask generator 844 can be further configured to exclude portion 910b when a trend of heartbeat data suggests that the sampling window 982 in which to accept data is during 920c after a time 940c when heartbeat is detected at 920a (i.e., the user is resting).

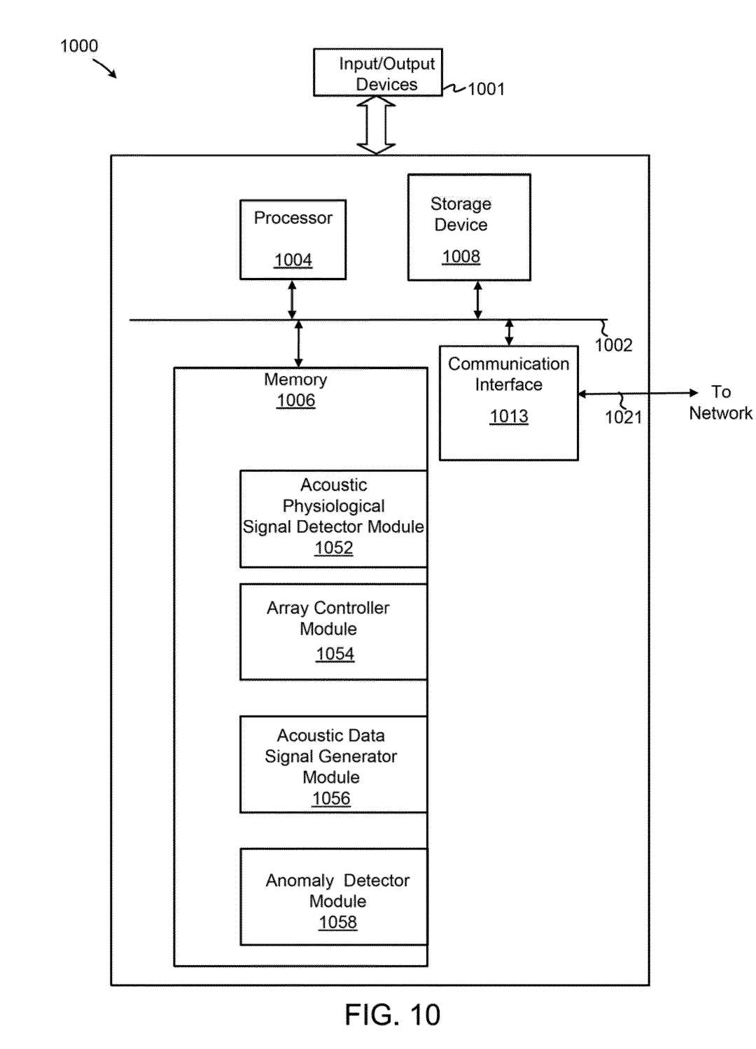

FIG. 10 illustrates an exemplary computing platform disposed in or used in association with a wearable device in accordance with various embodiments. In some examples, computing platform 1000 may be used to implement computer programs, applications, methods, processes, algorithms, or other software to perform the above-described techniques. Computing platform 1000 includes a bus 1002 or other communication mechanism for communicating information, which interconnects subsystems and devices, such as one or more processors 1004, system memory 1006 (e.g., RAM, etc.), storage device 1008 (e.g., ROM, etc.), a communication interface 1013 (e.g., an Ethernet or wireless controller, a Bluetooth controller, etc.) to facilitate communications via a port on communication link 1021 to communicate, for example, with a computing device, including mobile computing and/or communication devices with processors. Processor 1004 can be implemented with one or more central processing units (“CPUs”), such as those manufactured by Intel®Corporation, or one or more virtual processors, as well as any combination of CPUs and virtual processors. Computing platform 1000 exchanges data representing inputs and outputs via input-and-output devices 1001, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 1000 performs specific operations by processor 1004 executing one or more sequences of one or more instructions stored in system memory 1006, and computing platform 1000 can be implemented in a client-server arrangement, peer-to-peer arrangement, or as any mobile computing device, including smart phones and the like. Such instructions or data may be read into system memory 1006 from another computer readable medium, such as storage device 1008. In some examples, hard-wired circuitry may be used in place of or in combination with software instructions for implementation. Instructions may be embedded in software or firmware. The term “computer readable medium” refers to any tangible medium that participates in providing instructions to processor 1004 for execution. Such a medium may take many forms, including but not limited to, non-volatile media and volatile media. Non-volatile media includes, for example, optical or magnetic disks and the like. Volatile media includes dynamic memory, such as system memory 1006.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1002 for transmitting a computer data signal.

In some examples, execution of the sequences of instructions may be performed by computing platform 1000. According to some examples, computing platform 1000 can be coupled by communication link 1021 (e.g., a wired network, such as LAN, PSTN, or any wireless network) to any other processor to perform the sequence of instructions in coordination with (or asynchronous to) one another. Computing platform 1000 may transmit and receive messages, data, and instructions, including program code (e.g., application code) through communication link 1021 and communication interface 1013. Received program code may be executed by processor 1004 as it is received, and/or stored in memory 1006 or other non-volatile storage for later execution. In the example shown, memory 1006 can include various modules that include executable instructions to implement functionalities described herein. In the example shown, memory 1006 includes acoustic physiological signal detector module 1052, an array controller module 1054, an acoustic data signal generator module 1056, and anomaly detector module 1058.

Referring back to FIG. 1, wearable device 170 can be in communication (e.g., wired or wirelessly) with a mobile device 180, such as a mobile phone or computing device. In some cases, mobile device 180, or any networked computing device (not shown) in communication with wearable device 170 or mobile device 180, can provide at least some of the structures and/or functions of any of the features described herein. As depicted in FIG. 1 and other figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1 (or any subsequent figure) can represent one or more algorithms. Or, at least one of the elements can represent a portion of logic including a portion of hardware configured to provide constituent structures and/or functionalities.

For example, multimodal piezoelectric sensing device 200 of FIG. 2 and any of its one or more components, such as multimodal piezoelectric signal detector 221 and data signal generator 223, can be implemented in one or more computing devices (i.e., any mobile computing device, such as a wearable device or mobile phone, whether worn or carried) that include one or more processors configured to execute one or more algorithms in memory. Thus, at least some of the elements in FIG. 1 (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, multimodal piezoelectric sensing device 200 of FIG. 2 and any of its one or more components, such as multimodal piezoelectric signal detector 221 and data signal generator 223, can be implemented in one or more computing devices that include one or more circuits. Thus, at least one of the elements in FIG. 1 (or any subsequent figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. An apparatus comprising:
one or more multimodal physiological sensors configured to receive physiological signals in a first mode and to generate data communication signals in a second mode;
a wearable housing including the one or more multimodal physiological sensors, the wearable housing configured to position at least a subset of the one or more multimodal physiological sensors to receive a physiologic signal originating from human tissue; and
a multimodal physiological sensing device configured to receive a sensor signal to based on the physiologic signal, the multimodal physiological sensing device being further configured to generate data representing a physiological characteristic.

2. The apparatus of claim 1, wherein the multimodal physiological sensing device is configured to further to generate a heart rate signal as the physiological characteristic.

3. The apparatus of claim 1, wherein the one or more multimodal physiological sensors further comprise:
one or more multimodal piezoelectric transducers configured to receive acoustic physiological signals in the first mode and to generate acoustic communication signals in the second mode.

4. The apparatus of claim 3, wherein the acoustic signals are generated by either blood vessel pulsation or a human heart, or both.

5. The apparatus of claim 3, wherein the one or more multimodal piezoelectric sensors comprise:
a piezoelectric transducer configured to receive the acoustic physiological signals in an audible range of frequencies in the first mode.

6. The apparatus of claim 3, wherein the one or more multimodal piezoelectric sensors comprise:
a piezoelectric transducer configured to generate the acoustic communication signals in an ultrasonic range of frequencies in the second mode.

7. The apparatus of claim 3, wherein the one or more multimodal piezoelectric sensors comprise:
a piezoelectric transducer configured to receive the acoustic communication signals in an ultrasonic range of frequencies in a third mode.

8. The apparatus of claim 1, wherein the multimodal physiological sensing device comprises:
a physiological signal detector configured to determine a heart rate in the first mode; and
a data signal generator configured to generate a data communication signal in the second mode.

9. The apparatus of claim 1, wherein the one or more multimodal physiological sensors comprise:
a piezoelectric transducer configured to operate in the first mode and the second mode.

10. The apparatus of claim 9, wherein a portion of the piezoelectric transducer is formed external to a surface of the wearable housing, the portion of the piezoelectric transducer being configured to contact the human tissue.

11. The apparatus of claim 9, wherein the piezoelectric transducer is formed within the wearable housing, the wearable housing further comprising:
a first material having an acoustic impedance value in a range of acoustic impedance values including a value of acoustic impedance for the human tissue, the first material being disposed between an inner surface of the wearable housing and the piezoelectric transducer to facilitate propagation of an acoustic physiological signal from the human tissue to the piezoelectric transducer.

12. The apparatus of claim 9, wherein the piezoelectric transducer is formed within the wearable housing, the wearable housing further comprising:
a second material being disposed between an outer surface of the wearable housing and the piezoelectric transducer to facilitate propagation of an acoustic communication signal from the piezoelectric transducer to an environment external to the wearable housing.

13. The apparatus of claim 9, further comprising:
a coupler having an acoustic impedance equivalent to the human tissue, at least a first surface of the coupler being formed external to a surface of the wearable housing and second surface of the coupler being configured to communicate the acoustic signal from the first surface of the coupler to the piezoelectric transducer.

14. The apparatus of claim 1, wherein one or more multimodal physiological sensors comprise:
a skin surface microphone (“SSM”) being formed in the wearable housing to contact human tissue.

15. The apparatus of claim 1, wherein one or more multimodal physiological sensors comprise:
an array of piezoelectric transducers.

16. The apparatus of claim 15, further comprising:
a transducer selector configured to select a first subset of piezoelectric transducers to receive acoustic signals.

17. The apparatus of claim 15, further comprising:
an aberrant signal reducer configured to select a second subset of piezoelectric transducers to identify common acoustic signals in a first piezoelectric transducer and a second piezoelectric transducer, the aberrant signal reducer being further configured to identify the physiological signal as a difference between acoustic signals applied to the first piezoelectric transducer and the second piezoelectric transducer.

18. A method comprising:
receiving an acoustic signal originating from human tissue, the acoustic signal associated with a physiological characteristic;
generating a first piezoelectric signal responsive to the acoustic signal;
determining a portion of the piezoelectric signal associated with a heartbeat derived from the acoustic signal;
identifying a heart rate at a processor based on the portion of the piezoelectric signal;
detecting data to be transmitted acoustically; and
generating a second piezoelectric signal to transmit the data via a piezoelectric transducer to communicate the data.

19. The method of claim 17, wherein receiving the acoustic signal originating from the human tissue comprises:
receiving the acoustic signal via a coupler configured to communicate the acoustic signal from a surface of the human tissue to a piezoelectric sensor, the coupler having an acoustic impedance equivalent to the human tissue.

20. The method of claim 17, further comprising:
transmitting data representing the heart rate to a device.
